(19) United States
(12) Patent Application Publication
(10) Pub. No.: US 2003/0228290 A1
(43) Pub. Date: Dec. 11, 2003

(54) Modular assembly of tissue engineered constructs

(76) Inventors: Michael V. Sefton, Toronto (CA); Alison McGuigan, Toronto (CA)

Correspondence Address: RICHES, MCKENZIE & HERBERT, LLP, SUITE 1800, 2 BLOOR STREET EAST, TORONTO, ON, M4W 3J5, CA

(21) Appl. No.: 10/453,631

(22) Filed: Jun. 4, 2003

(30) Foreign Application Priority Data
Jun. 5, 2002 (CA) 2,389,394

(51) Int. Cl. A61K 45/00; C12N 5/08

(52) U.S. Cl. 424/93.7; 435/370

(57) ABSTRACT

A vascularised tissue construct seeded with endothelial cells is disclosed. A modular, scalable method of fabrication of tissue constructs with a uniform cell distribution that can accommodate multiple cell types, and in which the porosity is created after cell incorporation, is disclosed. The construct is based on the porous structure that is created when a column or tube is randomly packed with solid objects, such as cylindrical rods. Cells, such as liver cells, are encapsulated in solid gelatin rods (200 μm diameter, aspect ratio 1:5) on to which if desired endothelial cells, such as human umbilical vein endothelial cells (HUVEC), can adhere. The gelatin rods are randomly packed into a larger tube and then coated with HUVEC in one embodiment. The interstitial gaps among the rods form interconnected channels that are lined by the endothelial cells. The resulting endothelial cell lining enables whole blood to percolate around the rods and through the interstitial channels. Sufficient nutrient supply, oxygen supply, and waste removal from the encapsulated cells are provided, through whole blood percolates since the encapsulated cells are close to the ‘pseudo-capillaries’ formed in the tissue construct.

### FIELD OF THE INVENTION

This invention relates to modular, scalable, vascularised tissue constructs that can accommodate multiple cell types, methods of fabricating the same, and uses thereof.

### BACKGROUND OF THE INVENTION

It is desirable to create an unlimited supply of vital organs, such as hearts, livers and kidneys, for example, for transplantation through tissue engineering. In the past, there have been many suggested approaches to tissue engineering. One fundamental difficulty in creating large three-dimensional organs is the creation of a vascularised support structure in the engineered tissue.

The prior art suggests that cells may be grown in culture and seeded into a porous scaffold. Unfortunately, it is often difficult to get cells deposited on the outside of a scaffold to migrate to the interior; typically they populate just the periphery of the scaffold, at best an outer millimetre or so. The initial cell distribution is not uniform and prior seeding approaches work best for small, two-dimensional constructs, such as for use in small animals, as disclosed by Burg et al. (J Biomed Mater Res, September 15; 51(4):642-9, 2000). However, this method does not scale well for larger constructs or larger animals. Some effort has been directed towards various dynamic seeding techniques, as discussed, for example, by Kim et al. (Bioeng, January 5; 57(1):46-54, 1998) and Vunjak-Novakovic et al. (Biotechnol Prog, March-April; 14(2):193-202, 1998). However the scalability of dynamic seeding techniques remains questionable.

Another limitation of current seeding strategies is the difficulty of mixing two or more different cells types together without the concern that the faster growing cell type will overtake the slower one. Layering one cell type over a different cell type, as in collagen gel vascular grafts as reported by Weinberg et al. (Science, January 24; 231(4736):397-400, 1986), circumvents this problem, but this method is not universally applicable.

It has been proposed by Mooney et al. (J. Control Rel., 64:91-102, 2000) to incorporate an endothelial cell mitogen (in this case, vascular endothelial growth factor, or VEGF) into three-dimensional porous poly(lactide-co-glycolide) (PLG) scaffolds during fabrication to promote scaffold vascularisation. Sustained delivery of bioactive VEGF translated into a significant increase in blood vessel ingrowth in mice and the vessels appeared to integrate with the host vasculature, as disclosed by Peters et al. (Abstracts of the Third Biennial Meeting of the Tissue Engineering Society, Nov. 30-Dec. 3, 2000). However, as described by Ahrendt et al. (Tissue Engineering, 4(2):117-130, 1998), VEGF is but one angiogenic factor and issues associated with the functional maturity of the vessels and the need for multiple factors may limit this strategy.

Vacanti et al. (Tissue Engineering, 6:105-117, 2000) proposed a hierarchical branched network mimicking the vascular system in two dimensions. Vacanti et al. etched silicon and Pyrex* surfaces with branching channels ranging from 10 μm to 500 μm in diameter, which were then seeded with rat hepatocytes and microvascular endothelial cells. This technique has been extended to a degradable polymer and flow through the channels was demonstrated using fluorescent microbeads, as disclosed by Terai et al. (Abstracts of the Third Biennial Meeting of the Tissue Engineering Society, Nov. 30-Dec. 3, 2000). *—Trade Mark

The endothelium comprises heterogeneous, metabolically active cells. There are considerable phenotypic differences between large and small vessel endothelial cells (EC), among different organs and even within the same organ, as disclosed by Hewett et al. (In Vitro Cell Dev Biol Anim, November; 29A(11):823-30, 1993). For example, growth characteristics of large and small vessel EC of the same organ vary on gelatin, as shown by Beekhuizen et al. (J Vasc Res, July-August; 31(4):230-9, 1994). These differences reflect environment differences: the extracellular matrix, paracrine/autocrine factors, cell-cell contact and biomechanical factors act as cues. Several counteracting mechanisms and factors (for instance, endothelin/NO, pericytes/TGFβ) likely cooperate to further regulate the maintenance of a quiescent phenotype under non-pathological conditions.

In normal tissue, the EC that line blood vessels and capillaries have a variety of roles in controlling vascular function. Secretion and surface expression of molecules such as nitric oxide (Palmer et al., Nature, 333:664-666, 1988), prostacyclin (Moncada, British J Pharm, 76:3-31, 1982) and endothelin, that act on smooth muscle cells, regulate vessel tone while those acting on leukocytes such as platelet-activating factor (McIntyre et al., J Clin Invest, July; 76(1):271-80, 1985), direct both initiation and progression of inflammation. Endothelial cells provide a haemocompatible surface by production of molecules that modulate platelet aggregation (such as prostacyclin, ADPase, von Willebrand Factor, vWF), coagulation (such as thrombomodulin, which regulates protein C, and tissue factor) and fibrinolysis (such as tissue plasminogen activator, tPA and, plasminogen activator inhibitor, PAI-1). Under normal physiological conditions, the endothelium has a non-thrombogenic phenotype but, depending on the local environment, the cell can be transformed into a pro-thrombotic surface, for example by the action of thrombin.

Blood compatibility has been a consideration in the development of vascular grafts. Endothelial cells have been seeded on a variety of biologically compatible materials, with or without protein pre-coating with fibronectin, collagen and other proteins, as shown by Meinhart et al. (Ann Thorac Surg, 71:S327-31, 2001). Factors influential in the EC seeding technique have been identified as including cell source and isolation technique, method of cell deposition, EC adhesion to the graft under flow conditions, and the thrombogenicity of the EC, as described by Hedeman Joosten et al. (J Vasc Surg, 28:1094-1103, 1998). Pre-seeded cells may be lost on implantation due to insufficient adhesion, as shown by Williams (Cell Trans, 4(4):401-410, 1995), and thus the protection from thrombosis provided by the cells may be limited due to the incomplete cell coverage of the support structure.

Various strategies have been explored to improve cell adhesion, as disclosed by Lin et al. (Biomaterials, 13(13):905-14, 1992); for example, precoating with adhesive protein as described by Vohra et al. (Br J Surg, April; 78(4):417-20, 1991), Schneider et al. (Clin Mater, 13(1-4):51-5, 1993) and Jarrell et al. (J Biomech Eng, May; 113(2):120-2, 1991). It is desirable to ensure that the EC remain adherent to the support structure, such that there are no bare spots, and that the EC maintain their antithrombogenic phenotype for proper vascularisation.

It may be desirable to mix two or more different cell types together in a tissue construct. However, in a mixed-cell type tissue construct, current seeding strategies fail to adequately address the situation where a faster growing cell type will overtake a slower one. Accordingly, it is desirable to provide a tissue construct that allows for incorporation of two or more cell types that have different growth rates, and a means of fabricating the same.

### SUMMARY OF THE INVENTION

Accordingly, it is an object of this invention to at least partially overcome the disadvantages of the prior art. Also, it is an object of this invention to provide a new modular approach to the fabrication of tissue engineering constructs that is scaleable, with a largely uniform cell distribution, and can accommodate multiple cell types and in which the porosity is created after cell incorporation.

A further object of the invention is to create a vascularised tissue construct by seeding a construct containing smooth muscle cells with endothelial cells.

The present invention resides in the porous structure that is created when an enclosure, preferably a column or tube, is randomly packed with a suitable material that can operate as a surface to which cells may adhere (“packing”). Preferably, the modules are the shape of cylindrical rods. A random arrangement of modules results in interstitial spaces in the packing. A plurality of interstitial spaces are interconnected such that there are interconnected channels throughout the packing, which results in the packing being porous. The packing may be arranged in the same manner as random packed columns found in chemical engineering process equipment or in chromatography/gel filtration columns. Preferably, the packing is packed so that the channels in such columns are narrow, resulting in relatively high surface area and high mass transfer coefficients for a fluid that may pass through the porous packing (i.e., across the enclosure). Accordingly, such columns are efficient separating devices. This arrangement is advantageously adapted in the present invention for tissue engineering.

An embodiment of the invention provides for a tissue construct including biological cells that are preferably encapsulated in a suitable material selected to enable the adherence of other biological cells to its surface, forming a “module”. A plurality of modules is distributed inside an enclosure to form packing. A module may measure from 10 μm to 20 m, as measured along the longest axis of the module with the preferred lengths being 100 μm to 1 cm. The aspect ratio (length to lateral dimension) may vary from 1 to 1000 or even greater. The enclosure may measure from 0.1 mm to 1000 mm, as measured along the longest axis of the enclosure, and the preferred dimensions are 0.5 mm to 10 cm. The lateral dimension is critical and must be between about 100 μm to about 500 μm.

Preferably, the modules are a geometric shape selected to enable a packing of a predetermined porosity. Preferably, the packing will have a porosity of 0.3 to 0.99, where the porosity is defined as the ratio of the volume of interstitial space to the volume of the enclosure.

The modules preferably are additionally coated with cells such that interstitial spaces remain in the packing, which form interconnected channels that are lined by the coated cells. Preferably, the coated cells do not completely fill the interstitial spaces between the modules, and the resulting interconnected channels remain large enough to allow fluid flow through the channels. Accordingly, fluid may flow around the modules and across the enclosure, thereby allowing mass transfer between the fluid and the coated cells.

Although the modules are preferably packed in an enclosure such that a porous structure results, the porosity will not preserve cell viability if the tissue construct is beyond a size where diffusion distances become too large.

Larger tissue constructs may require an internal vascularised structure that preferably has nonthrombogenic surfaces. Accordingly, the tissue construct of the present invention may include endothelial cells coating the modules that result in a “pseudo-capillary” network (i.e. the interconnected channels) capable of supporting blood perfusion through the channels of the porous packing.

In a preferred embodiment of the invention, liver cells are encapsulated in gelatin rods or spheres, more preferably rods of 50 to 500 μm diameter and an aspect ratio of 5 to 1 (length to diameter), onto which endothelial cells, for example, human umbilical vein endothelial cells (HUVEC), can adhere. These gelatin rods or spheres are preferably randomly packed into a tube that may measure 1 mm to 1000 mm, as measured along the longest axis of the tube. The gelatin rods or spheres are preferably coated on their outside by HUVEC. The packing, which includes the gelatin rods or spheres and HUVEC coating the same, further includes interstitial spaces that are interconnected to form channels such that the packing is porous. Preferably, the porosity and channel size are sufficiently large to allow whole blood to percolate around the rods or spheres and through the channels.

Preferably, the gelatin rods or spheres include sufficient structural rigidity and strength to allow their packing in a tube without deformation of the rods or spheres and without allowing the encapsulated liver cells to grow beyond the boundaries of the rods or spheres.

In another embodiment, the invention includes randomly packed larger diameter rods proximal and distal to the “pseudo-capillary” bed in the enclosure. Accordingly, the invention provides for means to produce a more physiological branching hierarchy.

In a further embodiment, the invention resides in modules of encapsulated cells that consist of one or more mixtures of different cell types. Advantageously, because the modules prevent cell growth beyond their confines, encapsulation of multiple cell types is possible without compromising viability of the coating cells. Accordingly, the invention provides for multiple modules encapsulating different cells to form a mixed cell tissue construct.

In another embodiment, the encapsulated cells are genetically manipulated to enhance cell survival or function. Further, the cells may have specialised cell functions for example, HepG2 spheroids for ‘liver-like’ function.

In yet another aspect, the present invention provides a tissue construct that further includes the use of cross-linking agents to produce stiffer, easier to handle constructs, without compromising cell viability. Examples of cross-linking agents may include, but are not limited to, polyepoxide, carbodiimide, genpin or glutaraldehyde.

In yet another aspect, the present invention provides a tissue construct that further comprises the use of a biocompatible polymer that may provide more rigid and easier to handle modules, preferably without compromising cell viability. Examples of biocompatible polymer may include, but are not limited to, agarose, alginate, collagen, polyacrylates, synthetic polymer that is substantially stable in vivo, or degradable material such as gelatin.

In yet another aspect, the present invention provides for a method of manufacturing the tissue construct wherein the cells that coat the modules may be introduced to incomplete modules prior to their insertion into the enclosure. In an alternative embodiment, cells may be introduced into the enclosure to coat the modules after introduction of the incomplete modules into the enclosure. In either embodiment, the module is “complete” as having a first cell type encapsulated in a suitable material of a geometric shape, onto which a second cell type adheres to coat the geometric shape. However, an incomplete module may still be useful. The interstitial gaps in the packing (i.e., between the modules) form interconnected channels that are lined by cells, regardless of the method of cell introduction. The resulting cell lining preferably enable fluid flow around the modules and through these channels.

In the preferred embodiment, the gelatin modules may be first packed into the tube, and subsequently coated on their outside by HUVEC by seeding the tube with HUVEC. Alternatively, the gelatin modules may be first coated with HUVEC and then packed into the tube. The interstitial gaps among the rods or spheres form interconnected channels that become lined by the endothelial cells, regardless of the method of seeding.

The present invention also provides a new method for connecting the vascularised construct to a vascular system. Preferably, connection of the vascularised construct can be made by using a vascular graft as the enclosure or a combination of inlet and outlet grafts and a separate enclosure to hold the modules. More preferably, conventional anastomotic procedures and/or adopting techniques used in assembling hollow fibre systems into larger units are considered in the invention.

Further aspects of the invention will become apparent upon reading the following detailed description and drawings that illustrate the invention and preferred embodiments of the invention.

### BRIEF DESCRIPTION OF THE DRAWINGS

In the drawings, which illustrate embodiments of the invention:

FIG. 1(a) is a representation of liver cells in a gelatin rod coated with endothelial cells;

FIG. 1(b) is the representation of a tube packed with gelatin rods;

FIG. 2 is a representation of gelatin rod containing HepG2 spheriods, one day after crosslinking with glutaraldehyde for 20 minutes;

FIG. 3 shows cells after being exposed to glutaraldehyde;

FIG. 4 is a photograph of gelatin rods having been seeded with HUVEC after 2 hours;

### DETAILED DESCRIPTION OF THE PREFERRED EMBODIMENTS

Experiments were first conducted with the human liver hepatoma cell line (HepG2), in the form of spheroids, as model cells, in part because of the potential to fabricate vascularised modules with some liver-like function. Furthermore, monitoring the phenotype of these cells through urea synthesis, albumin secretion and antitrypsin release is relatively straightforward for such metabolically active cells. Also these cells are sensitive to co-culture with endothelial cells, to cytokines (eg IL6), to oxygen levels and to their 3-D configuration. The spheroidal arrangement was used as the starting point because under these conditions HepG2 cells are known to show increased activity over that of a monolayer culture.

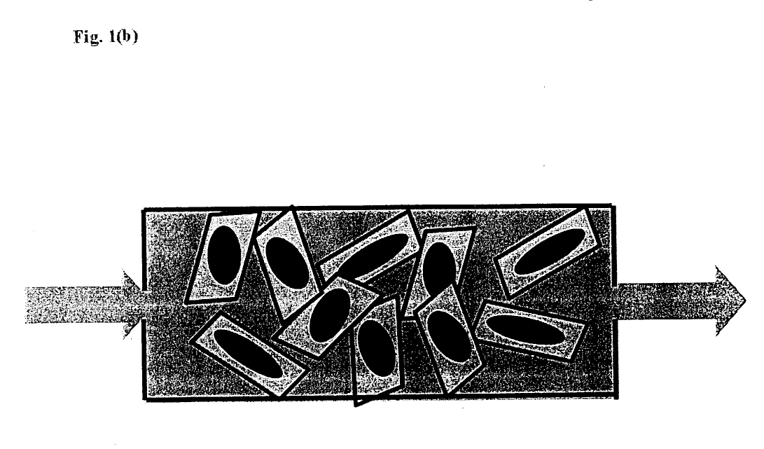

Gelatin rods (˜120 μm diameter×1 mm long) containing HepG2 spheroids were prepared inside a glass micropipette (0.282 mm ID, Drummond* microcap) prewashed with Pluronic* L101. HepG2 spheroids were prepared by culture in αMEM with serum on bacteriological polystyrene culture dishes for 4 days; at this time spheroids were approximately 100 μm in diameter and contained roughly 300 cells each. Spheroids were suspended in 55 μl of 300 bloom, type A gelatin (25 wt %) liquid (˜40° C.) and a droplet of the gel-spheroid suspension was placed onto a sterilised glass slide, from which it was drawn into the glass micropipette. After 20-30 minutes refrigeration, (enough time to ensure gelation) the gel-spheroid rods were expelled from the glass capillary into a sterile solution of very dilute glutaraldehyde (0.05%) in PBS. After 20 minutes the rods were washed twice in PBS followed by a 1-2 hour wash in cell culture medium. A 20 minute exposure time was sufficient to cross-link the gelatin so that the rods (FIG. 2, scale is 100 μm) did not fall apart when incubated at 37° C., yet avoided prolonged exposure of the cells to glutaraldehyde. The majority of the surfactant was easily removed from the gel rod surface by washing the rods in medium. Rods were cut by hand under the microscope into 1 mm lengths. *—Trade Mark

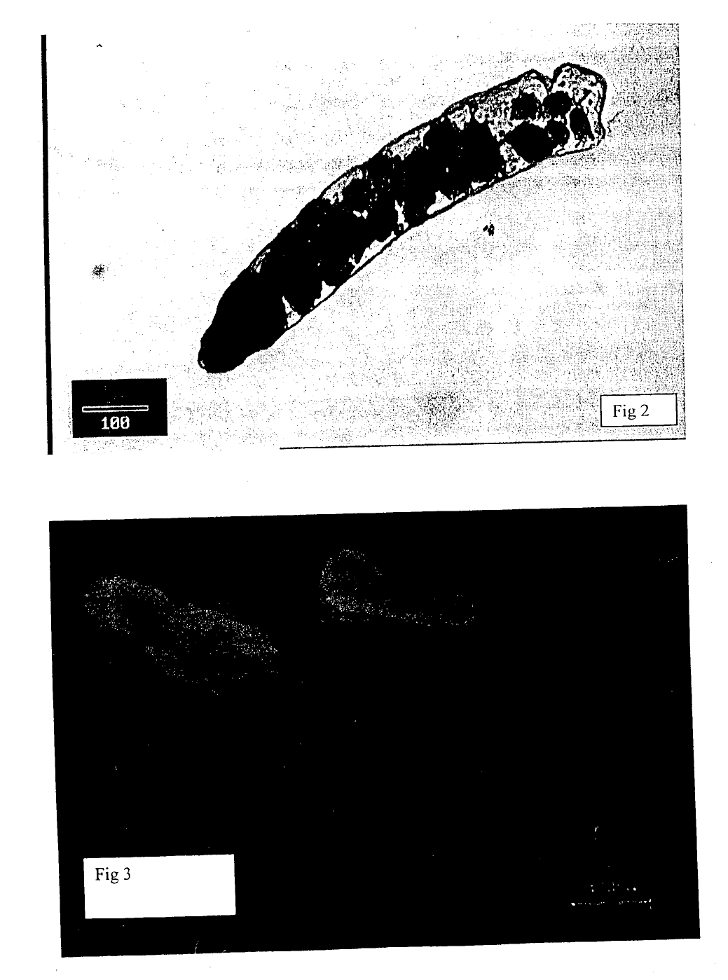

Despite the various manipulations and especially the brief exposure to very dilute glutaraldehyde, the cells appeared to remain largely viable based on MTT conversion (the spheroids became purple) and confocal microscopy with the live/dead cell assay (FIG. 3), at least for 9 days after fabrication. Not surprisingly the central core of the spheroids remained viable while the outer rim had dead cells, presumably reflecting the effect of the glutaraldehyde.

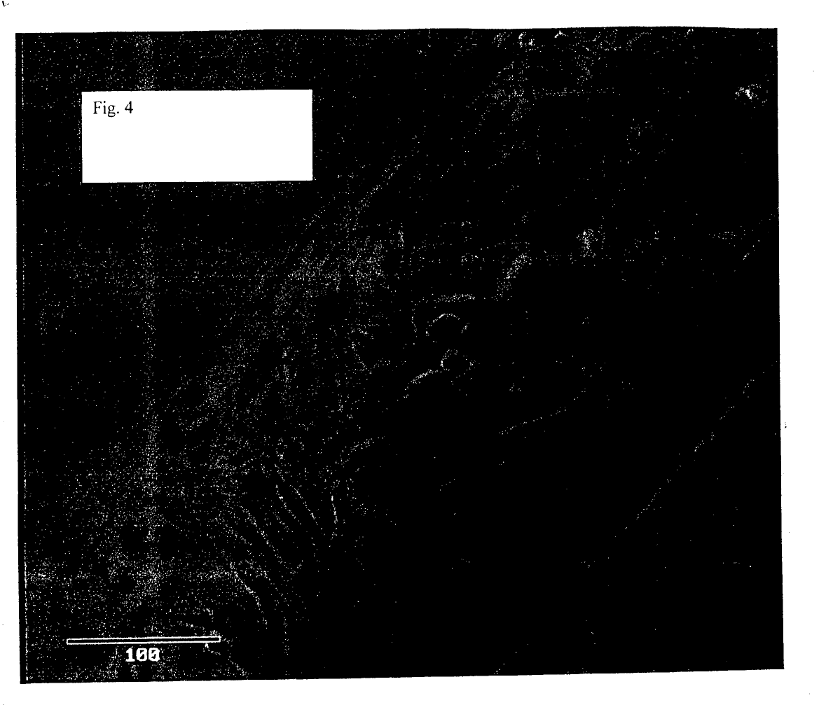

Gelatin rods (no HepG2) were seeded with HUVEC; the cells readily adhered as expected (FIG. 4, 2 hours). Rods were randomly packed without difficulty in 2 mm ID PE tubes, capped with a mesh.

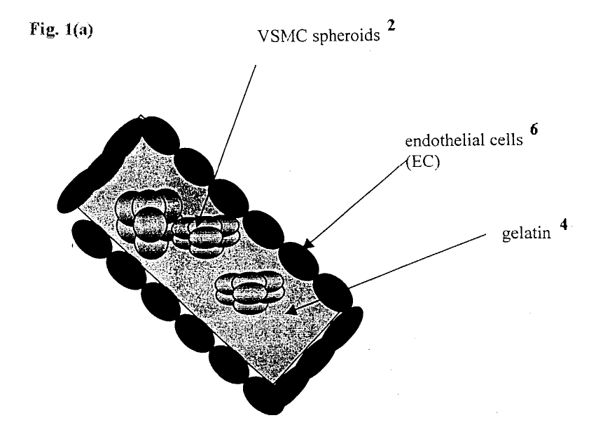

Cylindrical modules were chosen instead of simpler, spherical ones because of the greater porosity of randomly packed rods instead of spheres when placed inside a larger tube as shown in FIG. 1b. The effect of aspect ratio on packing density is well known in the fiber composite materials and chemical engineering literatures. At an aspect ratio, the (L/D) of 5 the porosity is ˜0.7, instead of the ˜0.5 that is obtained with spheres. This porosity is necessary to provide space for subsequent cell seeding, to lower the pressure drop across the rod packed tube, but most importantly to minimise the shear stress on the seeded endothelial cells. The fluid velocity (and hence shear stress) through a packed bed of rods is strongly dependent on the porosity (ε): the proportionality is to ε³/(1−ε)², according to the Ergun equation. Using 2 dynes/cm² as a maximum tolerable shear stress for endothelial cells, the maximum superficial velocity through a packed bed of rods of L/D=5 (ε˜0.7) is calculated to be 0.82 cm/s; if the bed were packed with spheres (ε=0.48 assuming a cubic lattice), the maximum velocity would be 0.22 cm/s. This difference translates into a 5 fold throughput advantage for the cylindrical modules: 154 ml/min/cm² of tube cross-section area for cylindrical modules, 41.8 ml/min/cm² for spherical modules. The corresponding pressure drops are 6.4 mm Hg and 15.4 mm Hg for a 10 cm long packed bed, respectively—well within the range that can be exploited in vivo.

FIG. 1(a) is a representation of a module showing liver cells 2 in a gelatin 4 rod coded with epithelial cells 6.

Packing gelatin rods into the PE tubing (the enclosure) is done by pouring a slurry of the gel rods suspended in PBS (phosphate buffered saline) into the larger diameter tube. A nylon mesh filter (Millipore*, pore size 100 μm) was used at the bottom of the tubing to retain the rods; a similar one was mounted on the top to create the tubing construct. *—Trade Mark

Endothelial cells were seeded onto the gelatin rods after loading the rods into the polyethylene tube much as vascular grafts are seeded. The assembled construct was filled with an EC suspension (2-4×10⁵ cells/cm² of gelatin or 1.2-2.5×10⁷ cells/cm³ for a 2 mm diameter×5 cm tube with 70% porosity) and the construct “soaked” in cell suspension for 2 hours to enable the EC to settle and adhere to the gelatin. After gentle rinsing, subsequent static culture for 1 to 4 days was sufficient to reach confluence. The soaking conditions (time, EC concentration) is optimised based on the number of cells retained by the gelatin rods and the uniformity of coverage.

Alternatively, EC is seeded on the gelatin rods prior to assembly into the PE tube (the enclosure) under static conditions in 24 well non-tissue culture plates (to minimise adhesion to the plate itself). The cells are allowed to adhere to the gelatin (1-4 hour incubation) and then cultured to reach confluence over 4 days. Some agitation of the rods within the 24 well plates is needed to obtain reasonably uniform coverage. These are then loaded into the PE tube as above; some loss of EC may occur during this loading step, necessitating a brief incubation (<1 day) to restore a monolayer.

Gelatin cylinders were molded in glass tubes using 25% gelatin (type A, 300 bloom, bovine skin, Sigma). Gelatin was sterilised by autoclaving for 20 minute at 120° C.

As stated hereinbefore, the characteristic dimension of the module is about 100 μm, such that the construct (most preferably with liver cells) is a better mimic of arteriole structure than capillary structure. Accordingly, good nutrient supply, oxygen supply and waste removal from the encapsulated cells is achieved by the vascularised construct, as the modules are preferably less than 100 μm away from a “pseudo-capillary”.

It will be understood to a person skilled in the art that the choice of geometric shape or size for the module will affect the fluid flow regime throughout the enclosure. For instance, pressure drop across the enclosure and shear forces to which the cells coating the modules are exposed will be influenced by the choice of geometric shape or size. For example, constructs consisting of randomly packed cylindrical rods result in greater porosity (and hence, lower pressure drop/shear forces) than would be obtained with spheres. Similarly, larger cylindrical rods will result in greater pore sizes than smaller cylindrical rods. Accordingly, the present invention includes the use of modules of any geometric shape or size to achieve the desired fluid flow characteristics throughout the enclosure.

Further, it may be desirable to use two or more different irregular and/or geometric shapes or sizes in combination, randomly distributed within in the enclosure, to achieve different porosities and flow regimes throughout the enclosure. Examples of geometric shapes may include, but are not limited to, cylinders, rods of hexagonal cross-section, rods of maltese cross-section, spheres, spheroids, ellipsoids, cones, conoids, tetrahedrons, cuboids, prisms, pyramids, frustums, wedges, toruses, toroids, hexahedrons, octahedrons, dodecahedrons, rhombohedrons and trapezohedrons.

It will be understood that, although various features of the invention have been described with respect to one or another of the embodiments of the invention, the various features and embodiments of the invention may be combined or used in conjunction with other features and embodiments of the invention as described and illustrated herein.

Although this disclosure has described and illustrated certain preferred embodiments of the invention, it is to be understood that the invention is not restricted to these particular embodiments. Rather, the invention includes all embodiments that are functional, electrical or mechanical equivalents of the specific embodiments and features that have been described and illustrated herein.

The embodiments of the invention in which an exclusive property or privilege is claimed are defined as follows:

1. A tissue construct having a porous structure created by a plurality of cell-containing modules randomly distributed therein.

2. The tissue construct as claimed in claim 1 wherein said porous structure is vascularized.

3. The tissue construct as claimed in claim 2 wherein said module comprises:
a core comprising one or more cell types;
a semi-permeable coating material over the said core, the coating material consisting of a biocompatible polymer; and
additional cells adhering to said biocompatible polymer.

4. The tissue construct of claim 3 wherein the biocompatible polymer is selected from the group consisting of agarose, alginate, collagen, gelatin, polyacrylates, a synthetic polymer that is substantially stable in vivo, and degradable material.

5. The tissue construct of claim 3, wherein said adhering cells consist of endothelial cells.

6. The tissue construct of claim 3, wherein said at least one cell type consists of liver cells.

7. The tissue construct of claim 3, wherein said at least one cell type comprises two or more different cell types.

8. The tissue construct of claim 3, wherein said core additionally consists of a biocompatible support matrix.

9. The tissue construct of claim 8, wherein said biocompatible support matrix is a gel.

10. The tissue construct of claim 8, wherein the biocompatible support matrix or the biocompatible coating material additionally contains a crosslinking agent.

11. The tissue construct of claim 10, wherein said cross-linking agent is selected from the group consisting of polyepoxide, carbodiimide, genpin and glutaraldehyde.

12. The tissue construct of claim 3, wherein said module comprises a major axis and a minor axis, wherein a ratio of said major axis to said minor axis is 5 to 1.

13. The tissue construct of claim 3, wherein said module has a shape selected from the group consisting of cylindrical rods, rods of hexagonal cross-section, rods of maltese cross-section, sphere, spheroid, ellipsoid, cone, conoid, tetrahedron, cuboid, prism, pyramid, frustum, wedge, torus, toroid, hexahedron, octahedron, dodecahedron, rhombohedron and trapezohedron.

14. The tissue construct of claim 3, wherein said plurality of modules comprise at least two modules of a different shape or size.

15. A module comprising:
a core containing cellular material consisting of cells and cell aggregates, said cells comprising at least one cell type, and
a semi-permeable coating material over the core, the coating material consisting of a biocompatible polymer and adhering cells to said biocompatible polymer.

16. The tissue construct as claimed in claim 1 wherein different modules contain different cell types.

17. A tissue construct as claimed in claim 1 wherein each module comprises a core containing cellular material consisting of cells and cell aggregates, said cells comprising at least one cell type, and
a semi-permeable coating material over the core, the coating material consisting of a biocompatible polymer and adhering cells to said biocompatible polymer.

18. A tissue construct as claimed in claim 2 wherein each module comprises a core containing cellular material consisting of cells and cell aggregates, said cells comprising at least one cell type, and
a semi-permeable coating material over the core, the coating material consisting of a biocompatible polymer and adhering cells to said biocompatible polymer.
